(19) United States
(12) Patent Application Publication
(10) Pub. No.: US 2024/0185955 A1
(43) Pub. Date: Jun. 6, 2024

(54) METHOD FOR GENERATING AN ENHANCED HI-C MATRIX, NON-TRANSITORY COMPUTER READABLE MEDIUM STORING A PROGRAM FOR GENERATING AN ENHANCED HI-C MATRIX, METHOD FOR IDENTIFYING A STRUCTURAL CHROMATIN ABERRATION IN AN ENHANCED HI-C MATRIX, AND METHODS FOR DIAGNOSING AND TREATING A MEDICAL CONDITION OR DISEASE

(71) Applicant: CHROMATINTECH BEIJING CO, LTD, Beijing (CN)

(72) Inventors: Yueying HE, Beijing (CN); Yue XUE, Beijing (CN); Jingyao WANG, Beijing (CN); Yiqin GAO, Beijing (CN)

(73) Assignee: CHROMATINTECH BEIJING CO, LTD, Beijing (CN)

(21) Appl. No.: 17/796,446

(22) PCT Filed: Nov. 23, 2021
(86) PCT No.: PCT/CN2021/132559

§ 371 Date: Jul. 29, 2022

(51) Int. Cl. G16B 30/10; G16B 20/30

(52) U.S. Cl. CPC G16B 30/10; G16B 20/30

(57) ABSTRACT

A method for generating an enhanced Hi-C matrix, a non-transitory computer readable medium storing a program for generating an enhanced Hi-C matrix, a method for identifying a structural chromatin aberration in an enhanced Hi-C matrix, and methods for diagnosing and treating a medical condition or disease. The method for generating an enhanced Hi-C matrix includes denoising an input Hi-C matrix to obtain a balanced distance matrix, denoising the balanced distance matrix to obtain a denoised distance matrix, sorting and ranking the denoised distance matrix to obtain a ranked distance matrix, calculating an adjacency matrix based on the ranked matrix, and calculating Laplacian eigenmaps of the adjacency matrix to obtain an enhanced Hi-C matrix.

### TECHNICAL FIELD

Embodiments of this application relates to a method for generating an enhanced Hi-C matrix, a non-transitory computer readable medium storing a program for generating an enhanced Hi-C matrix, a method for identifying a structural chromatin aberration in an enhanced Hi-C matrix, and methods for diagnosing and treating a medical condition or disease such as cancer.

### BACKGROUND

High-throughput chromosome conformation capture (Hi-C) allows for genome-wide profiling of chromatin interactions in space and has been used to study the genome-wide interactions of genomes. It is well known that spatial organization of chromatin is non-random and is crucial for deciphering how the 3D architecture of DNA affects genome functionality and transcription. Hi-C technology provides a deeper insight into the 3D organization of chromatin by comprehensive detection of spatial interactions between genomic regions. Hi-C technology typically involves the production of hundreds of millions of paired-end sequencing reads. It can capture chromatin interactions across an entire genome and construct a genome-wide Hi-C contact matrix, where each element in the matrix denotes the contact strength between any two regions of genome.

A “contact” is a read pair that remains after reads that do not align uniquely to the genome, that correspond to unligated fragments, or that are duplicates are excluded, as disclosed in as discussed in US 2017/0362649 to Lieberman-Aiden et al., which is hereby incorporated by reference. The contact matrix can be visualized as a heatmap, whose entries are called “pixels”. An “interval” refers to a (one-dimensional) set of consecutive loci; the contacts between two intervals thus forming a “rectangle” or “square” in the contact matrix. “Matrix resolution” is defined as the locus size used to construct a particular contact matrix and “map resolution” as the smallest locus size such that a certain threshold of loci have a certain threshold of contacts. The map resolution describes the finest scale at which one can reliably discern local features in the data. FIG. 1, for example, illustrates a conventional contact matrix, where each pixel represents the contact frequency between a 1-Mb locus and another 1-Mb locus.

In other words, Hi-C technology measures interaction frequency between loci, and not distance per se. Typically, formaldehyde is used to initiate crosslinking between loci. Formaldehyde crosslinking will occur only between loci which physically interact. Thus, a weak Hi-C signal between two loci indicates that the interaction occurred in a small fraction of the population. In order to determine the distance between the two loci, simplifying assumptions about how interaction frequencies relate to physical distances must be made.

Bioinformatics tools including algorithms, computational, and statistical methods have been used for the exploration and interpretation of Hi-C data. These pipelines cover all current aspects of Hi-C analysis workflow, ranging from preprocessing of sequencing reads to normalization and inference of genome structure. The preprocessing pipeline consists of read mapping, fragment assignment, filtering and binning, and we are left with a symmetrical contact matrix. Each entry in the matrix reflects the interaction frequency observed between the corresponding pair of loci (i.e., bins). The two loci are separated by a fixed size genomic interval, which is conveyed as the resolution. Following preprocessing, normalization is carried out to correct systematic biases, making Hi-C samples more comparable and downstream analysis reliable. The inference of genome architecture can then be investigated at different levels, such as topologically associating domains (TADs). TADs are regarded as functional and structural units of higher-order spatial genome organization of many eukaryotic genomes.

In mammalian genomes, 5 types of patterns are typically observed in Hi-C matrices: (1) cis/trans interaction ratio, (2) distance-dependent interaction frequency, (3) genomic compartments, (4) chromatin rings and TADs, and (5) point interactions. Researchers have developed a series of algorithms to capture chromatin rings and TADs, examples of which are shown in FIG. 2.

FIGS. 3 and 4 illustrate how a Hi-C heatmap can be analyzed to find chromatin rings and TAD structure. See Eagen, K., “Principles of Chromosome Architecture Revealed by Hi-C,” Trends Biochem Sci., 43(6), pp. 469-478, June 2018, and available at: https://www.ncbi.nlm.nih.gov/pmc/articles/PMC4347522/, which is hereby incorporated by reference. As seen in FIG. 3, the strength of each pixel indicates the relative, pair-wise contact probability of two loci. TADs are on-diagonal boxes of contact enrichment. Rings or loops are radially symmetric peaks of contact intensity, often located at the corners of TADs in mammalian cells. Off-diagonal boxes indicate interactions due to compartmentation. FIG. 4 illustrates chromatin rings and TADs. Compartmentation is indicated by homotypic (active-active or inactive-inactive) TAD-TAD interactions.

The raw Hi-C matrix without any treatment will be affected by systematic biases, including technical biases from sequencing and mapping, that affect the reliability of downstream interpretations. Other factors, such as selection of enzymes, treatment time and the number of cells used will affect the results, so it is not possible to directly compare Hi-C matrix among different biological samples.

Normalization techniques have been developed to remove unwanted systematic biases and are one of the most important pipelines in Hi-C data analysis. Normalization attempts to remove the unwanted systematic biases, so that the interaction frequencies reflecting the underlying architecture can be preserved as far as possible. Conventional Hi-C normalization methods included sequential component normalization (SCN), HiCNorm, iterative correction and eigenvector decomposition (ICE), Knight-Ruiz (KR), chromoR and multiHiCcompare.

By analyzing Hi-C data, researchers have noticed that the chromatin spatial structure varies among cell types. But conventional normalization methods are difficult to analyze effectively and lack reliability. In this regard, corrected HiC matrices from these methods from similar samples (for instance, samples derived from a same cancer type) still display diverse characteristics. FIGS. 5 and 6, for example, display a HiC matrix normalized by ICE for cancer cells of the same type (FIG. 5) and normal cells of the same type (FIG. 6) normalized by a known method. As seen in FIGS. 5 and 6, it is difficult to discern similarities across samples.

Historically, the main approaches finding 3D structural changes in cancerous process focus on local specific interaction, i.e., existing methods focus on finding structural variations (SVs) sites, which are caused by changes in one-dimensional sequence, including deletion, translocation, replication, and so on. But during carcinogenesis, chromatin structures change globally such that identification of local changes alone is incomplete, non-transferable. Hi-C technology provides one possible avenue for better identification of chromatin structures change globally.

Accurately finding the location with structural changes in aberrant cells is important for diagnosis and treatment of medical conditions or disease with a genetic basis such as cancer. By looking for specific chromatin interactions that exist only in cancer or only in normal cells, potential locus associated with cancer can be identified. Therefore, there is a significant need in bioinformatics for methods that are useful in identifying chromatin structure and differences between structures in normal versus aberrant cells. These and other problems are addressed by the following disclosed embodiments.

### SUMMARY

The inventors found that by looking for a broader range of structural change and better defined hotspots using disclosed embodiments it is possible to more reliably and more efficiently find the difference in chromatin structure between different types of cells. They also found that such methods could be very useful in diagnosing and treating a myriad of medical conditions or disease including, but not limited to, cancer. According to disclosed embodiments, Hi-C matrices generated from different sources, different sequence depths and different cell counts are comparable in a novel and surprisingly effective manner.

In a first embodiment, there is provided a method for generating an enhanced Hi-C matrix. The method includes denoising an input Hi-C matrix to obtain a balanced distance matrix, denoising the balanced distance matrix to obtain a denoised distance matrix, sorting and ranking the denoised distance matrix to obtain a ranked distance matrix, calculating an adjacency matrix based on the ranked matrix, and calculating Laplacian eigenmaps of the adjacency matrix to obtain an enhanced Hi-C matrix.

In another embodiment, there is provided a non-transitory computer readable medium storing a program for generating an enhanced Hi-C matrix. The program causes the processor to execute denoising an input Hi-C matrix to obtain a balanced distance matrix, denoising the balanced distance matrix to obtain a denoised distance matrix, sorting and ranking the denoised distance matrix to obtain a ranked distance matrix, calculating an adjacency matrix based on the ranked matrix, and calculating Laplacian eigenmaps of the adjacency matrix to obtain an enhanced Hi-C matrix.

In another embodiment, there is provided a method for identifying a structural chromatin aberration in an enhanced Hi-C matrix. The method includes providing target cells and normal cells, generating an enhanced Hi-C matrix according to disclosed methods for each of the target cells and the normal cells, and analyzing the enhanced Hi-C matrices to identify a structural chromatin aberration in the target cells.

In another embodiment, there is provided a method for diagnosing a medical condition or disease. The method includes identifying a structural chromatin aberration according to disclosed methods, and relating the structural chromatin aberration to a medical condition or disease.

In another embodiment, there is provided a method for treating a medical condition or disease. The method includes identifying a structural chromatin aberration according to disclosed methods, and administering a gene therapy vector to a subject in need thereof. The structural chromatin aberration is indicative of a medical condition or disease.

### BRIEF DESCRIPTION OF THE DRAWINGS

To describe the technical solutions in embodiments of the present invention or in the prior art more clearly, the following briefly introduces the accompanying drawings needed for describing the embodiments or the prior art. Apparently, the accompanying drawings in the following description illustrate merely some embodiments of the present invention, and persons of ordinary skill in the art may still derive other drawings from these accompanying drawings without creative effort.

FIG. 1 and FIG. 2 illustrate a raw contact Hi-C matrix heatmap (FIG. 1) and a chromatin ring and TADs visual plot (FIG. 2) generated according to known methods.

FIG. 3 and FIG. 4 illustrate a sample Hi-C matrix analysis showing correspondence of a heatmap (FIG. 3) to schematic representation of the chormatin (FIG. 4).

FIG. 5 and FIG. 6 illustrate normalized contact Hi-C matrix heatmaps for cancer cells (FIG. 5) and normal cells (FIG. 6) normalized by a known method.

FIG. 7 is a schematic illustration of a method for generating an enhanced Hi-C matrix according to an embodiment.

FIG. 8 is a schematic illustration of sub-steps of a method for generating an enhanced Hi-C matrix according to an embodiment.

FIG. 9 is a schematic illustration of sub-steps of a method for generating an enhanced Hi-C matrix according to an embodiment.

FIG. 10 is a schematic illustration of sub-steps of a method for generating an enhanced Hi-C matrix according to an embodiment.

FIG. 11 is a schematic illustration of sub-steps of a method for generating an enhanced Hi-C matrix according to an embodiment.

FIG. 12 and FIG. 13 illustrate normalized contact Hi-C matrix heatmaps for cancer cells (FIG. 12) and normal cells (FIG. 13) normalized by a method according to an embodiment.

FIG. 14 and FIG. 15 illustrate Laplacian eigenmaps for cancer cells (FIG. 14) and normal cells (FIG. 15) normalized by a method according to an embodiment.

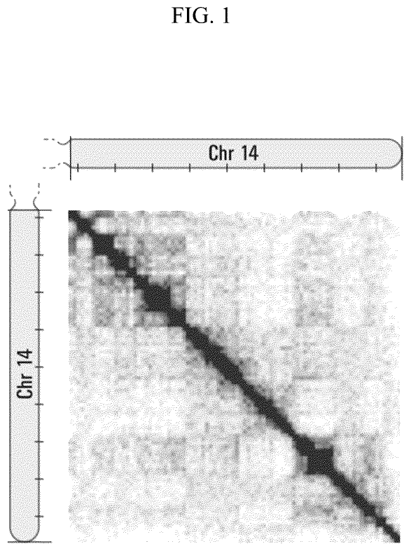

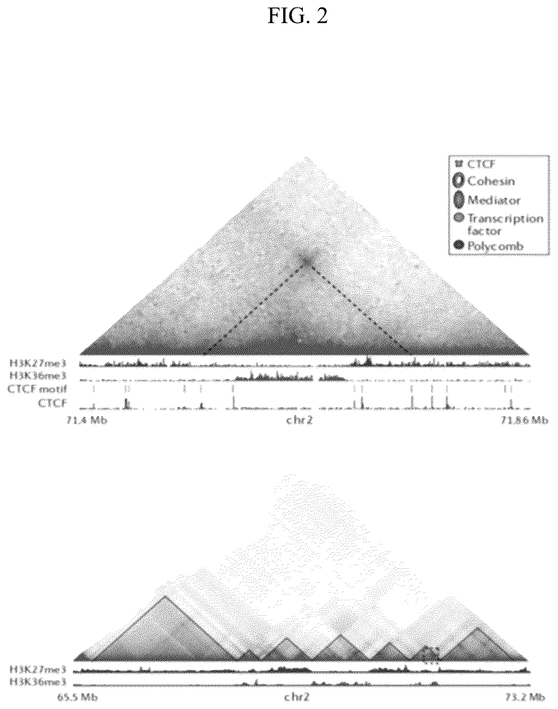

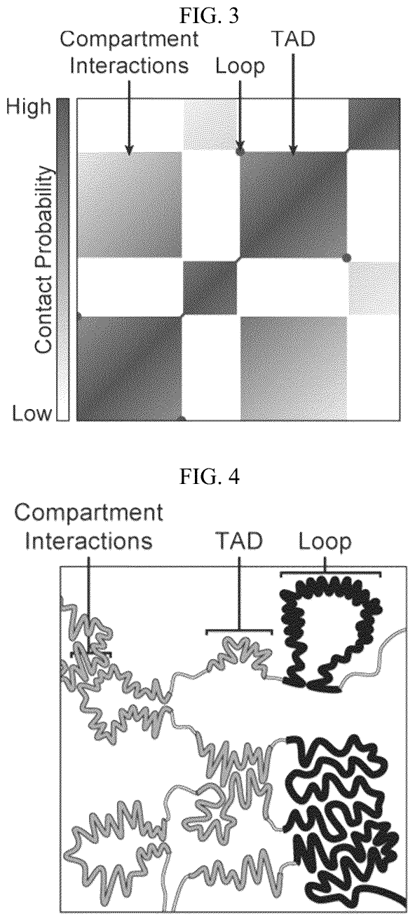

### DESCRIPTION OF EMBODIMENTS

To make the objectives, technical solutions, and advantages of embodiments of the present invention clearer, the following clearly and comprehensively describes the technical solutions in embodiments of the present invention with reference to the accompanying drawings in embodiments of the present invention. Apparently, the described embodiments are merely a part rather than all embodiments of the present invention. All other embodiments obtained by persons of ordinary skill in the art based on embodiments of the present invention without creative effort shall fall within the protection scope of the present invention.

Disclosed embodiments enhance Hi-C data analysis and characterize the 3D structural changes of chromatin rather than by being limited to local features. Disclosed embodiments perform global embedding and dimension reduction on Hi-C data to visualize the chromatin structure and extract 3D structural features or changes during biological processes. Disclosed embodiments further allow for the identification of variable loci in the targeting and treatment of a medical condition or disease, such as cancer. Treatment may involve the usage of transcription or translation production of the obtained loci as a medical condition or disease target.

### Methods for Generating an Enhanced Hi-C Matrix

Hi-C data produced by deep sequencing is similar to other genome-wide deep sequencing datasets. The data starts out as genomic reads in the traditional FASTQ file format (containing a DNA read string and a phred quality (QV) score string). Data storage requirements for Hi-C datasets are guided by the sequencing depth needed to attain a desired resolution and the size of the FASTQ files. The processed Hi-C data will normally be order(s) of magnitude smaller than the size of the FASTQ files. The FASTQ file is then processed according to known methods in the art that include read mapping, fragment assignment, fragment filtering, binning, bin level filtering, balancing, and analysis/interpretation

The so-called “matrix” is formed in the binning step. In this step, bins (i.e., rows/columns) are formed so that the data can be stored in a fixed-size symmetrical matrix format. Conventionally, in the balancing step, one attempts to balance the matrix by any number of known ways. This step is based on the assumption that since the goal is to view the entire interaction space in an unbiased manner, each fragment/bin should be observed approximately the same number of times. Typically, an algorithm is then applied iteratively until convergence. It is important to visually assess the data before and after bias correction, in order to determine if the procedure was successful. A successful filtering and bias correction would smooth the interaction matrix such that no obviously high rows/columns would remain. Disclosed embodiments are directed to significant advances in these and other methods for generating an enhanced Hi-C matrix.

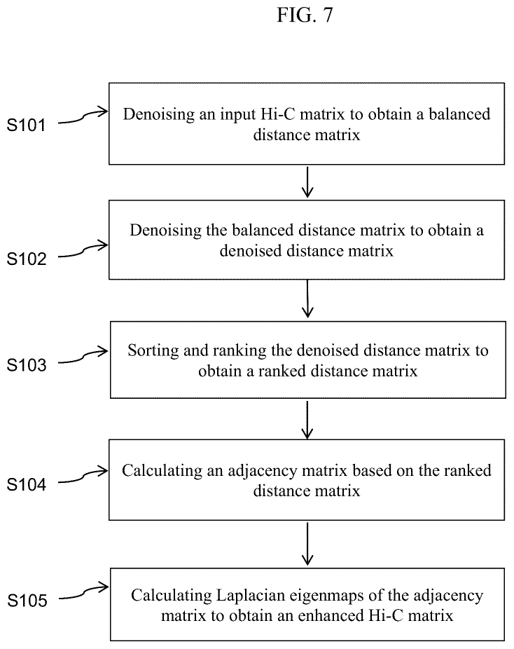

With reference to FIG. 7, denoising is performed on a Hi-C matrix to obtain a balanced distance matrix in step S101. In embodiments, the denoising step employs a network denoising algorithm. The network denoising algorithm may include, but is not limited to, a Diffusion State Distance (DSD) algorithm. A DSD algorithm is a network denoising algorithm based on the random walk theory. In the context of bioinformatic modeling, DSD is a convergence metric on the vertices of a graph. Previous results on the convergence of DSD to a limiting metric relied on the definition being based on symmetric or reversible random walk on the graph. Convergence has been shown to hold even when the DSD is based on general finite irreducible Markov chains.

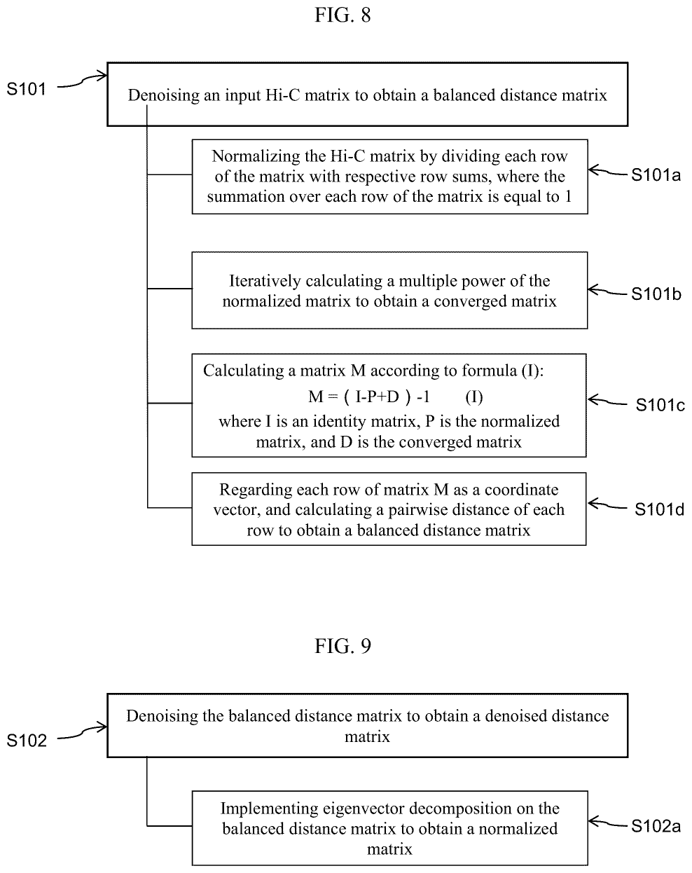

The denoising step S101 according to embodiments may include normalizing the Hi-C matrix by dividing each row of the matrix with respective row sums, where the summation over each row of the matrix is equal to 1, to obtain a normalized matrix in step S101a, as seen in FIG. 8. Alternatively, the Hi-C matrix may already be normalized by methods known in the art. Such methods include, but are not limited to, SCN, HiCNorm, ICE, KR, chromoR, and multiHiCcompare.

A multiple power of the normalized matrix may be iteratively calculated to obtain a converged matrix in step S101b. Then, in step S101c, a matrix M may be calculated according to formula (I) below:

M=(I−P+D)−1 (I)

where I is an identity matrix, P is the normalized matrix, and D is the converged matrix.

Next, each row of matrix M may be regarded as a coordinate vector, and pairwise L1 distance of each row may be calculated to obtain a balanced distance matrix in step S101d.

Further denoising is then further performed on the balanced distance matrix to obtain a denoised distance matrix in step S102. This step may include implementing eigenvector decomposition on the balanced distance matrix in step S102a, as seen in FIG. 9. The eigenvector vector is the vector that responds to a matrix as though that matrix were a scalar coefficient, i.e., axes along which linear transformation acts. The first eigenvalue (sorted by absolute value) is set to zero, and the denoised distance matrix is calculated.

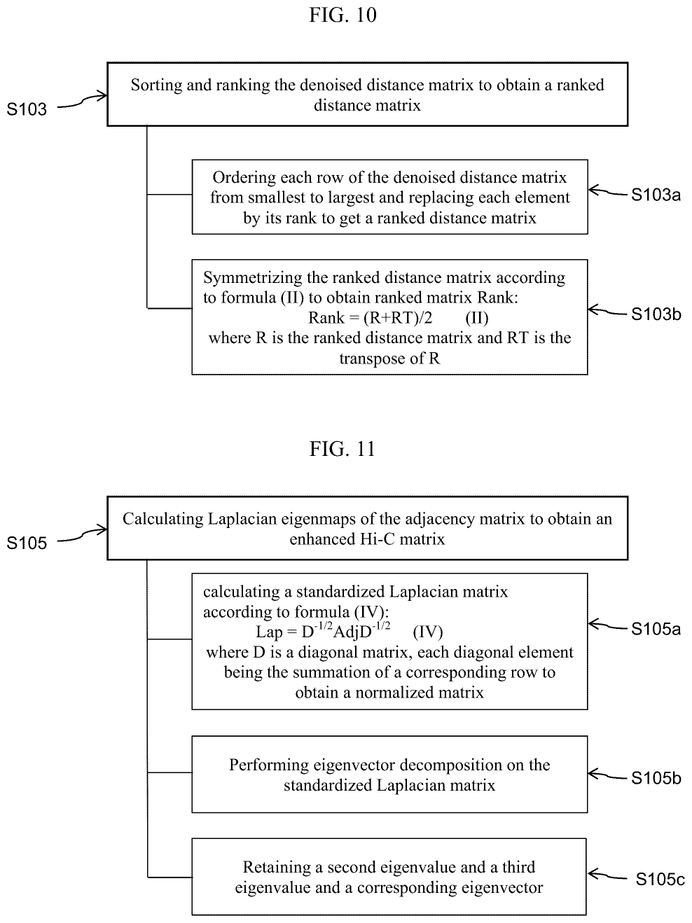

In step S103, sorting is then performed on the denoised distance matrix and each element is replaced by its rank to obtain a ranked distance matrix. This step may include ordering each row of the denoised distance matrix from smallest to largest and replacing each element by its rank to get a ranked distance matrix in step S103a, as seen in FIG. 10. In step S103b, the ranked distance matrix may then be symmetrized according to formula (II) below to obtain ranked matrix Rank:

Rank=(R+RT)/2 (II)

where R is the ranked distance matrix and RT is the transpose of R.

In step S104, an adjacency matrix Adj is calculated based on the ranked matrix according to formula (III) below:

Adj=e−Rank/σ (III)

where σ can be any positive number.

In step S105, Laplacian eigenmaps of the adjacency matrix Adj are calculated. Laplacian eigenmaps correspond to Euclidean distances between nearby points that are transformed to similarity scores (to be used as weights). As seen in FIG. 11, this step may include, in step S105a, calculating the standardized Laplacian matrix according to formula (IV) below:

Lap=D−½AdjD−½ (IV)

where D is a diagonal matrix, each diagonal element being the summation of a corresponding row.

Eigenvector decomposition may then performed on the standardized Laplacian matrix in step S105b. In step S105c, the second and third eigenvalue and the corresponding eigenvector may then be retained.

The result of the above method is an enhanced genome-wide interaction matrix, i.e., the enhanced Hi-C matrix, where each entry reflects an interaction frequency between two genomic loci. The enhanced Hi-C matrix allows for the finding of a changeable structural hotspot or hotspot contact in the genome by comparing 3D chromatin structures between contrasting samples, e.g., cancer and normal cells.

Disclosed embodiments allow for the definition of the nearest n (50<n<500) chromatin loci of a corresponding locus as its neighbors. By comparing the neighbors of each locus between cancer and normal samples in the enhanced Hi-C matrix, it is possible to locate chromatin loci with a great change in neighbors, i.e., structural hotspots. The structural hotspots or hotspot-related contacts are helpful for the diagnosis and treatment of medical conditions or disease, including cancer. In this manner, the inventors have found specific genes that are highly correlated cancer. These include, but are not limited to, SPAG9, TOB1, and UTP18.

The disclosed method for generating an enhanced Hi-C matrix will now be described with respect to the following sample 3×3 contact matrix for further understanding of the disclosed embodiments. However, the disclosure is not intended to be limited to 3×3 contact matrices or the specific sample described below. It will be understood that the disclosed methods will be suitable for application to any Hi-C dataset.

In embodiments, the following operations are exemplified by the sample 3×3 contact Hi-C matrix illustrated below:

$$HiC = [ \begin{matrix}25 & 10 & 5 \\ 10 & 30 & 15 \\ 5 & 15 & 20\end{matrix} ]$$

To the above Hi-C matrix, the DSD algorithm is performed to obtain the distance matrix Dist. This process may include:
(1) Normalizing the Hi-C matrix by dividing each row with respective row sums to obtain the normalized matrix P, the summation over each row of P is equal to 1:

$$P = [ \begin{matrix}0 \text{.625} & 0.25 & 0.125 \\ 0.182 & 0 \text{.545} & 0.273 \\ 0.125 & 0 \text{.375} & 0.5\end{matrix} ] .$$

(2) Iteratively calculating the multiple power of P until converging to D:

$${P}^{2} = P * P = [ \begin{matrix}0.4517 & 0.339 & 0.209 \\ 0.247 & 0.445 & 0.308 \\ 0.209 & 0.423 & 0.368\end{matrix} ]$$

$${P}^{3} = {P}^{2} * P = [ \begin{matrix}0.331 & 0.392 & 0.276 \\ 0.286 & 0.412 & 0.302 \\ 0.276 & 0.415 & 0.309\end{matrix} ]$$

$${P}^{4} = {P}^{3} * P = [ \begin{matrix}0.298 & 0.406 & 0 \text{.295} \\ 0.296 & 0.407 & 0.296 \\ 0.295 & 0.408 & 0.297\end{matrix} ]$$

$${P}^{5} = {P}^{4} * P = [ \begin{matrix}0.296 & 0.407 & 0.296 \\ 0.296 & 0.407 & 0.296 \\ 0.296 & 0.407 & 0.296\end{matrix} ] = {P}^{6} = {P}^{7} = … … = D$$

(3) Calculating M=(I−P+D)−1:

$$\begin{matrix}{M = {( I - P + D )}^{- 1} = [ \begin{matrix}1 & 0 & 0 \\ 0 & 1 & 0 \\ 0 & 0 & 1\end{matrix} ] - [ \begin{matrix}0.625 & 0 \text{.250} & 0.125 \\ 0.182 & 0.545 & 0.273 \\ 0.125 & 0.375 & 0.5\end{matrix} ] + \\ [ \begin{matrix}0.296 & 0.407 & 0.296 \\ 0.296 & 0.407 & 0.296 \\ 0.296 & 0.407 & 0.296\end{matrix} ] )}^{- 1} \\ = [ \begin{matrix}1.625 & - 0.283 & - 0.341 \\ - 0.206 & 1.197 & 0.009 \\ - 0.341 & 0.013 & 1.329\end{matrix} ]\end{matrix}$$

(4) Regarding each row of matrix M as a coordinate vector, and calculating pairwise L1 distance (i.e., the absolute value of the component wise difference between the pixel and the class) of each row to get distance matrix Dist:

$$M = [ \begin{matrix}1.625 & - 0.283 & - 0.341 \\ - 0.206 & 1.197 & 0.009 \\ - 0.341 & 0.013 & 1.329\end{matrix} ] = [ \begin{matrix}c 1 \\ c 2 \\ c 3\end{matrix} ]$$

$$Dist = [ \begin{matrix}L 1 ( c 1 , c 1 ) & L 1 ( c 1 , c 2 ) & L 1 ( c 1 , c 3 ) \\ L 1 ( c 2 , c 1 ) & L 1 ( c 2 , c 2 ) & L 1 ( c 2 , c 3 ) \\ L 1 ( c 3 , c 1 ) & L 1 ( c 3 , c 2 ) & L 1 ( c 3 , c 3 )\end{matrix} ] = [ \begin{matrix}0 & 3.662 & 3.932 \\ 3.662 & 0 & 2.639 \\ 3.932 & 2.639 & 0\end{matrix} ]$$

To the above balanced matrix Dist, denoising is performed to get the denoised distance matrix Dist1. This process may include:
(1) Implementing eigenvector decomposition on matrix Dist:

$$Dist = {UVU}^{T} = [ \begin{matrix}0.617 & 0.784 & 0.067 \\ 0.547 & - 0.367 & - 0.752 \\ 0.565 & - 0.501 & 0.656\end{matrix} ] [ \begin{matrix}6.85 & 0 & 0 \\ 0 & - 4.224 & 0 \\ 0 & 0 & - 2.626\end{matrix} ] [ \begin{matrix}0.617 & 0.547 & 0.565 \\ 0.784 & - 0.367 & - 0.501 \\ 0.067 & - 0.752 & 0.656\end{matrix} ]$$

(2) Setting the first eigenvalue (sorted by absolute value) to zero, the denoised distance matrix Dist1=UV′UT:

$$\begin{matrix}Dist 1 = {UV}^{′} {U}^{T} = [ \begin{matrix}0.617 & 0.784 & 0.067 \\ 0.547 & - 0.367 & - 0.752 \\ 0.565 & - 0.501 & 0.656\end{matrix} ] [ \begin{matrix}0 & 0 & 0 \\ 0 & - 4.224 & 0 \\ 0 & 0 & - 2.626\end{matrix} ] [ \begin{matrix}0.617 & 0.547 & 0.565 \\ 0.784 & - 0.367 & - 0.501 \\ 0.067 & - 0.752 & 0.656\end{matrix} ] \\ = [ \begin{matrix}- 2.608 & 1.347 & 1.543 \\ 1.347 & - 2.054 & 0.519 \\ 1.543 & 0.519 & - 2.188\end{matrix} ]\end{matrix}$$

To the above denoised matrix Dist1, sorting is performed and each element is replaced by its ranks to obtain the ranked distance matrix Rank. This process may include:
(1) Ordering each row of Dist1 from smallest to largest and replacing each element by its rank to get matrix R:

$$R = [ \begin{matrix}1 & 2 & 3 \\ 3 & 1 & 2 \\ 3 & 2 & 1\end{matrix} ]$$

(2) Symmetrizing the ranked distance matrix R to obtain Rank=(R+RT)/2, where RT is the transpose of R:

$$Rank = ( R + {R}^{T} ) / 2 = ( [ \begin{matrix}1 & 2 & 3 \\ 3 & 1 & 2 \\ 3 & 2 & 1\end{matrix} ] + [ \begin{matrix}1 & 3 & 3 \\ 2 & 1 & 2 \\ 3 & 2 & 1\end{matrix} ] / 2 = [ \begin{matrix}1 & 2.5 & 3 \\ 2.5 & 1 & 2 \\ 3 & 2 & 1\end{matrix} ]$$

To the above ranked distance matrix Rank, the adjacency matrix Adj, Adj=e−Rank/σ, were σ can be any positive number and is set to 1 is performed, as in the following example:

$$Adj = {e}^{- Rank / σ} = [ \begin{matrix}0.368 & 0.092 & 0.050 \\ 0.082 & 0.368 & 0 \text{.135} \\ 0.05 & 0 \text{.135} & 0.368\end{matrix} ]$$

To the above adjacency matrix Adj, Laplacian eigenmaps are calculated. This process may include:
(1) calculating the standardized Laplacian matrix Lap=D−½AD−½, where D is a diagonal matrix, each diagonal element being the summation of a corresponding row:

$$Lap = {D}^{- 1 / 2} {AD}^{- 1 / 2} = [ \begin{matrix}1.414 & 0 & 0 \\ 0 & 1.307 & 0 \\ 0 & 0 & 1.345\end{matrix} ] [ \begin{matrix}0.368 & 0.092 & 0.050 \\ 0.082 & 0.368 & 0 \text{.135} \\ 0.05 & 0 \text{.135} & 0.368\end{matrix} ] [ \begin{matrix}1.414 & 0 & 0 \\ 0 & 1.307 & 0 \\ 0 & 0 & 1.345\end{matrix} ] = [ \begin{matrix}0.736 & 0.152 & 0.095 \\ 0.152 & 0.628 & 0.238 \\ 0.095 & 0.238 & 0.665\end{matrix} ]$$

(2) performing eigenvector decomposition on Lap, and retaining the second and third eigenvalue and the corresponding eigenvector.

### Methods for Identifying a Structural Chromatin Aberration in an Enhanced Hi-C Matrix

In another embodiment, there is provided a method for identifying a structural chromatin aberration in an enhanced Hi-C matrix providing target cells and normal cells. The method includes generating an enhanced Hi-C matrix according to the embodiment described above for each of the target cells and the normal cells. The method includes analyzing the enhanced Hi-C matrices to identify a structural chromatin aberration in the target cells.

The method may further include identifying at least one locus associated with the structural chromatin aberration in the target cells. The at least one locus may include, but is not limited to, SPAG9, TOB1, and UTP18.

### Methods for Diagnosing and Treating a Medical Condition or Disease

In other embodiments, there are provided methods for diagnosing and treating a medical condition or disease. The methods include identifying the structural chromatin aberration described above. In the method of diagnosing a disease, the structural chromatin aberration is indicative of a disease. In the method of treating a disease, the method includes administering a gene therapy vector to a subject in need thereof. The gene therapy may include usage of transcription or translation production of at least one locus associated with the structural chromatin aberration in the target cells as a disease target.

According to the disclosed methods, it is possible to identify regulatory genes or regulatory elements capable of modulating open reading frame sequences through physical interactions (close spatial proximity) between these regulatory elements and these open reading frames. The regulatory elements and open reading frame can be located near or far apart along the linear genome sequence or can be located on different chromosomes. The open reading frame sequences may be associated with a medical condition or disease.

In particular, it is possible to find the loci that are prone to change in medical condition or disease such as, for example, cancer, as the target of disease diagnosis and treatment. The inventors found that different types of cancer samples show highly consistent characteristics, indicating that this method is surprisingly effective in identifying the common characteristics of cancer cell structure, and providing new ideas for cancer diagnosis and treatment.

Disclosed embodiments are applicable to and operable on any medical condition or disease with a genetic basis. In this regard, the medical condition or disease may include, but is not limited to, cancer, cardiovascular disease, kidney disease, autoimmune disease, pulmonary disease, liver disease, lymphoid disease, bone marrow disease, bone disease, blood disorder, and the like.

### Non-Transitory Computer Readable Medium and Machine Learning

Disclosed embodiments further include a non-transitory computer readable medium storing a program for generating an enhanced Hi-C matrix, the program causing the processor to execute the disclosed methods. Disclosed embodiments may further include a variety of machine learning algorithms implemented on specialized computers or computer systems for executing any one or more of the disclosed methods. In this regard, the algorithms may be used for automatically executing steps using commercial or open source tools. Machine learning algorithms may be used for mathematically processing large genomic datasets and may also be used in optimizing calculations and increasing the precision and accuracy of outputs.

As is understood in the art of bioinformatics, machine learning algorithms involve establishing classifiers and training datasets. Classifiers play an important role in the analysis of complex multi-dimensional systems, such as chromatin structures and eukaryotic genomes. To develop classifications, supervised learning technology may be based on decision trees, on logical rules, or on other mathematical techniques such as linear discriminant methods (including perceptrons, support vector machines, and related variants), nearest neighbor methods, Bayesian inference, neural networks, and the like.

The programmatic tools used in developing the disclosed machine learning algorithms are not particularly limited and may include, but are not limited to, open source tools, rule engines such as Hadoop®, programming languages including SAS®, SQL, R, Matlab, and Python and various relational database architectures. In embodiments, Python is the preferred programming construct within which to execute disclosed methods.

The specialized computer or processing system that may implement disclosed methods and machine learning algorithms may be a specialized processing system and may be operational with numerous other general purpose or special purpose computing system environments or configurations, as would be understood by a bioinformatics practitioner. Examples of well-known computing systems, environments, and/or configurations that may be suitable for use with disclosed methods may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

Neural networks may be employed in executing disclosed methods. The neural network may be a deep convolutional neural network. The neural network may be a deep neural network that comprises an output layer and one or more hidden layers. In embodiments, training the neural network may include training the output layer by minimizing a loss function given the optimal set of assignments, and training the hidden layers through a backpropagation algorithm.

The deep neural network may be a Convolutional Neural Network (CNN). In a CNN-based model, a set of filters are used to extract features using convolution operation. Training of the CNN is done using a training dataset, which determines the trained values of the parameters/weights of the neural network.

In some CNN models, the numbers of the CNN layers and fully connected layers may vary. In some network architectures, residual pass or feedbacks may be used to avoid a conventional problem of gradient vanishing in training the network weights. The network may be built using any suitable computer language such as, for example, Python or C++. Deep learning toolboxes such as TensorFlow, Caffe, Keras, Torch, Theano, CoreML, and the like, may be used in implementing the network. These toolboxes are used for training the weights and parameters of the network. In some embodiments, custom-made implementation of CNN and deep learning algorithms on special computers with Graphical Processing Units (GPUs) are used for training, inference, or both. The inference is referred to as the stage in which a trained model is used to infer/predict the testing samples. The weights of a trained model are stored in a computer disk and then used for inference. Different optimizers such as the Adam optimization algorithm, and gradient descent may be used for training the weights and parameters of the networks. In training the networks, hyperparameters may be tuned to achieve higher recognition and detection accuracies. In the training phase, the network may be exposed to the training data through several epochs. An epoch is defined as an entire dataset being passed only once both forward and backward through the neural network.

The network can be trained using a transfer learning mechanism. In transfer learning, the network's weights are initially trained using a dataset different than the target dataset to learn the relevant features. Then, this pre-trained network is retrained further using the features in the target database. The CNN architecture can be 3D to handle 3D chromatin structural data.

### EXAMPLES

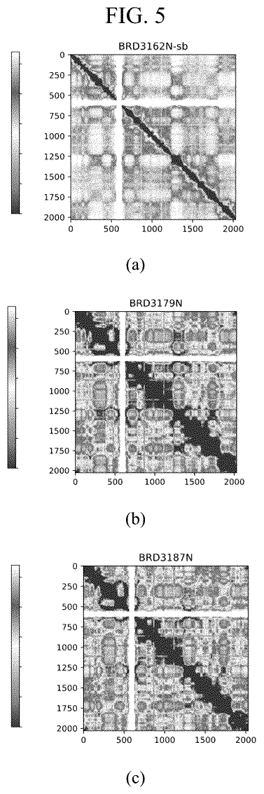

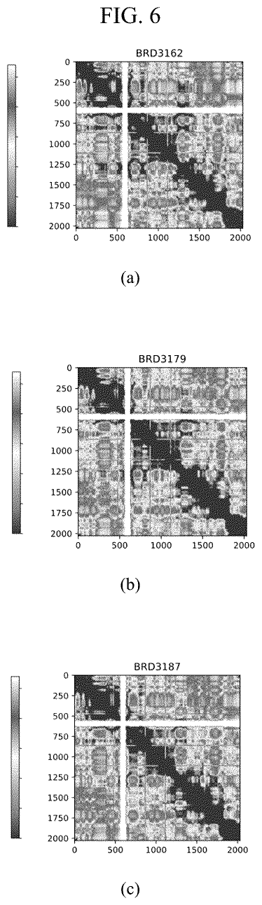

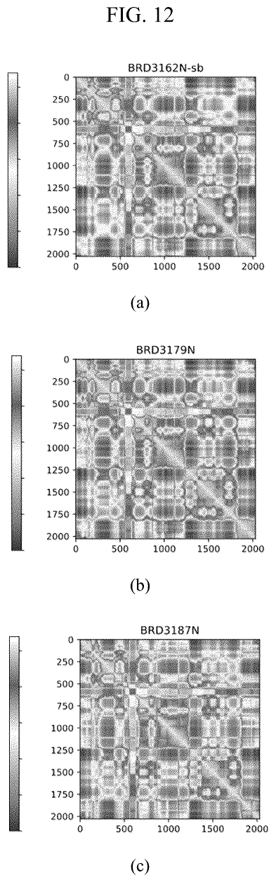

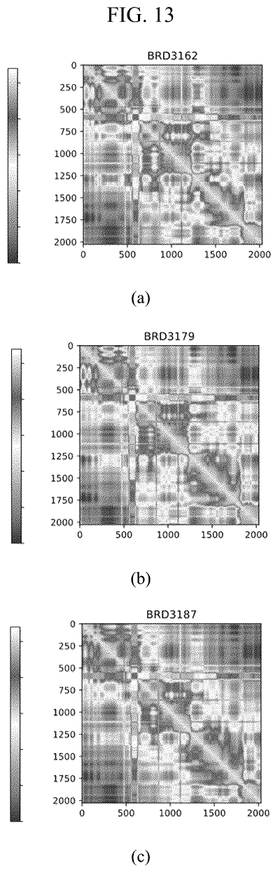

Cells from the same samples as shown in FIGS. 5 and 6 were processed. A Hi-C matrix of the cells was enhanced according to disclosed methods. The results of this enhancement are illustrated in FIGS. 12 and 13.

As seen in FIGS. 12 and 13, similar samples (each row) contain more similar characteristics, indicating that the structural information extracted from the Hi-C data by the disclosed methods is more reliable and effective than conventional methods, as seen in FIGS. 5 and 6. That is, the Hi-C matrix treated by disclosed methods is more comparable and conservative, and the difference of chromatin structure between different types of cells can be easily obtained.

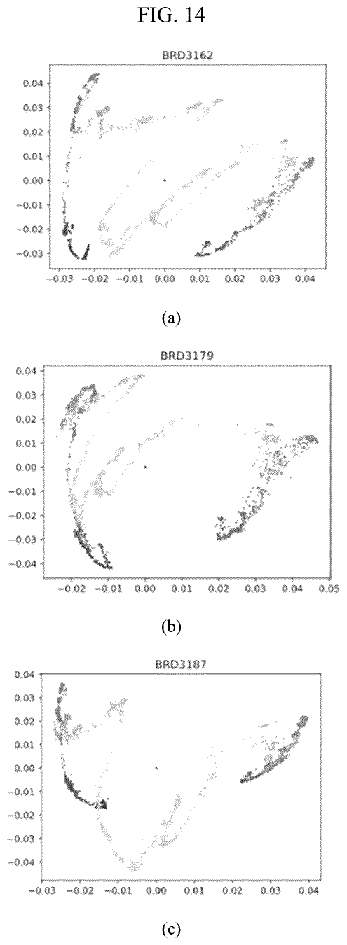

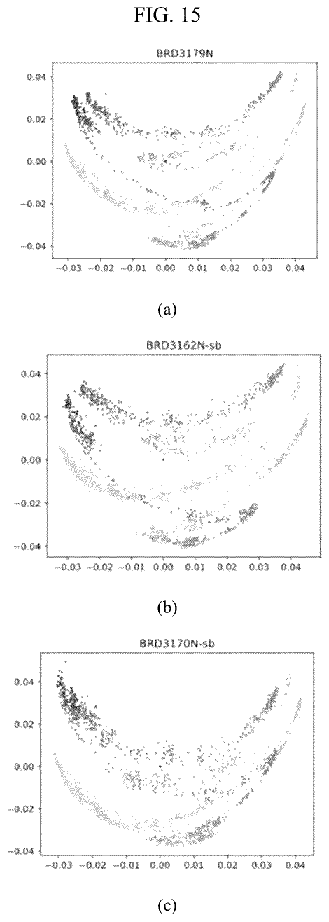

FIGS. 14 and 15 illustrate Laplacian eigenmaps for the same samples as in FIGS. 12 and 13. Each scatter plot in FIGS. 14 and 15 represents a 40 kb locus. As seen in FIGS. 14 and 15, the normal samples were packed tightly while the cancer samples were not. Thus, it was easy to distinguish the 3D structure of cancer samples from the normal samples in a global view.

It will be appreciated that the above-disclosed features and functions, or alternatives thereof, may be desirably combined into different devices, systems, and methods. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is:

1. A method for generating an enhanced Hi-C matrix, the method comprising:
denoising an input Hi-C matrix to obtain a balanced distance matrix;
denoising the balanced distance matrix to obtain a denoised distance matrix;
sorting and ranking the denoised distance matrix to obtain a ranked distance matrix;
calculating an adjacency matrix based on the ranked matrix; and
calculating Laplacian eigenmaps of the adjacency matrix to obtain an enhanced Hi-C matrix.

2. The method for generating the enhanced Hi-C matrix according to claim 1, wherein the input Hi-C matrix is a raw-data Hi-C matrix.

3. The method for generating the enhanced Hi-C matrix according to claim 1, wherein the input Hi-C matrix is a normalized Hi-C matrix generated by at least one of SCN, HiCNorm, ICE, KR, chromoR, and multiHiCcompare.

4. The method for generating the enhanced Hi-C matrix according to claim 1, wherein the step of denoising the Hi-C matrix to obtain the balanced distance matrix includes employing a Diffusion State Distance algorithm.

5. The method for generating the enhanced Hi-C matrix according to claim 1, wherein the step of denoising the Hi-C matrix to obtain the balanced distance matrix comprises:
normalizing the Hi-C matrix by dividing each row of the matrix with respective row sums, where the summation over each row of the matrix is equal to 1, to obtain a normalized matrix;
iteratively calculating a multiple power of the normalized matrix to obtain a converged matrix;
calculating a matrix M according to formula (I):
M=(I−P+D)−1 (I)
where I is an identity matrix, P is the normalized matrix, and D is the converged matrix; and
regarding each row of matrix M as a coordinate vector, and calculating a pairwise distance of each row to obtain a balanced distance matrix.

6. The method for generating a normalized a Hi-C matrix according to claim 1, wherein the step of denoising the balanced distance matrix to obtain the denoised distance matrix includes implementing eigenvector decomposition on the balanced distance matrix.

7. The method for generating a normalized a Hi-C matrix according to claim 1, wherein sorting and ranking the denoised distance matrix to obtain the ranked distance matrix comprises:
ordering each row of the denoised distance matrix from smallest to largest and replacing each element by its rank to get a ranked distance matrix; and
symmetrizing the ranked distance matrix according to formula (II) to obtain ranked matrix Rank:
Rank=(R+RT)/2 (II)
where R is the ranked distance matrix and RT is the transpose of R.

8. The method for generating a normalized a Hi-C matrix according to claim 1, wherein the adjacency matrix is calculated according to formula (III):
Adj=e−Rank/σ (III)
where σ is a positive number.

9. The method for generating a normalized a Hi-C matrix according to claim 1, wherein calculating Laplacian eigenmaps of the adjacency matrix to obtain the enhanced Hi-C matrix comprises:
calculating a standardized Laplacian matrix according to formula (IV):
Lap=D−½AdjD−½ (IV)
where D is a diagonal matrix, each diagonal element being the summation of a corresponding row;
performing eigenvector decomposition on the standardized Laplacian matrix; and
retaining a second eigenvalue and a third eigenvalue and a corresponding eigenvector.

10. The method for generating the enhanced Hi-C matrix according to claim 1, wherein a resolution of the enhanced Hi-C matrix is such that in a range of 50 to 500 neighbor loci are observable for each loci.

11. A non-transitory computer readable medium storing a program for generating an enhanced Hi-C matrix, the program causing the processor to execute:
denoising an input Hi-C matrix to obtain a balanced distance matrix;
denoising the balanced distance matrix to obtain a denoised distance matrix;
sorting and ranking the denoised distance matrix to obtain a ranked distance matrix;
calculating an adjacency matrix based on the ranked matrix; and
calculating Laplacian eigenmaps of the adjacency matrix to obtain an enhanced Hi-C matrix.

12. A method for identifying a structural chromatin aberration in an enhanced Hi-C matrix, the method comprising:
providing target cells and normal cells;
generating an enhanced Hi-C matrix according to the method of claim 1 for each of the target cells and the normal cells; and
analyzing the enhanced Hi-C matrices to identify a structural chromatin aberration in the target cells.

13. The method for identifying the structural chromatin aberration according to claim 12, further comprising identifying at least one locus associated with the structural chromatin aberration in the target cells.

14. The method for identifying the structural chromatin aberration according to claim 13, wherein the least one locus is selected from the group consisting of SPAG9, TOB1, and UTP18.

15. A method for diagnosing a medical condition or disease, comprising:
identifying a structural chromatin aberration according to the method of claim 12; and
relating the structural chromatin aberration to a medical condition or disease.

16. The method for diagnosing a medical condition or disease according to claim 15, wherein the medical condition or disease is selected from the group consisting of cancer, cardiovascular disease, kidney disease, autoimmune disease, pulmonary disease, liver disease, lymphoid disease, bone marrow disease, bone disease, and blood disorder.

17. A method for treating a medical condition or disease, the method comprising:
identifying a structural chromatin aberration according to claim 12; and
administering a gene therapy vector to a subject in need thereof,
wherein the structural chromatin aberration is indicative of a medical condition or disease.

18. The method for treating a medical condition or disease according to claim 17, wherein the gene therapy includes usage of transcription or translation production of at least one locus associated with the structural chromatin aberration in the target cells as a medical condition or disease target.

19. The method for treating a medical condition or disease according to claim 17, wherein the medical condition or disease is selected from the group consisting of cancer, cardiovascular disease, kidney disease, autoimmune disease, pulmonary disease, liver disease, lymphoid disease, bone marrow disease, bone disease, and blood disorder.

20. The method for treating a medical condition or disease according to claim 19, wherein the medical condition or disease is cancer.
